(19) United States
(12) Patent Application Publication
(10) Pub. No.: US 2006/0025328 A1
(43) Pub. Date: Feb. 2, 2006

(54) Compositions suitable for controlled release of the hormone GnRH and its analogs

(76) Inventor: Patrick J. Burns, Lexington, KY (US)

Correspondence Address: THOMAS P. MCCRACKEN;DURECT CORPORATION, 10240 BUBB ROAD, CUPERTINO, CA, 95014, US

(21) Appl. No.: 11/090,117

(22) Filed: Mar. 24, 2005

Related U.S. Application Data
(63) Continuation of application No. 09/336,328, filed on Jun. 18, 1999, now abandoned.
Application No. 09/336,328 is a continuation-in-part of application No. 09/001,123, filed on Dec. 30, 1997, now Pat. No. 6,051,558.
(60) Provisional application No. 60/047,789, filed on May 28, 1997.

(51) Int. Cl. A61K 38/22

(52) U.S. Cl. 514/2

(57) ABSTRACT

A liquid composition for the controlled release of gonodotropin releasing hormone (GnRH) or its analogs or agonists is provided that includes: (i) a non-polymeric, non-water soluble liquid carrier material (HVLCM) of viscosity of at least 5,000 cP at 37° C. that does not crystallize neat under ambient or physiological conditions; and (ii) GnRH or analogs agonists thereof. The composition can be used to treat reproductive conditions and/or induce ovulation in animals, such as livestock, fish, and shellfish.

This application claims priority to U.S. Ser. No. 09/001,123, filed on Dec. 30, 1997, the entire contents of which are hereby incorporated by reference, which claims priority to U.S. Provisional Application No. 60/047,789 filed on May 28, 1997.

### BACKGROUND OF THE INVENTION

In animal husbandry, the management of fertility can be both difficult and extremely important for the success of agricultural or other businesses. Stimulation of ovulation at appropriate times, as well as the induction of cyclicity in some species of domesticated animals that can become seasonally nonovulatory would result in increased management efficiency for these species.

For example, in the husbandry of horses, the development of an accurate, economical method for the precise control of ovulation in the mare would greatly benefit reproductive management of mares and stallions. The mares' extended estrus period, with ovulation at any time from 1 to 10 days after the beginning of estrus, has made reproductive management of mares time-consuming, expensive and most importantly, inefficient. In the mare, GnRH or its analogs are beginning to be used as alternative non-antigenic substitutes to replace hCG to hasten ovulation in preovulatory mares. This is because repeated use of hCG has been associated with decreased response [Sullivan, J., J. Am. Vet. Med. Assoc. 63:895(1973)] and anti-hCG antibody formation [Roser, J., J. Reprod. Fert. Suppl. 173-179(1974)]. Current data suggest that ovulation induction with potent GnRH analogs requires multiple injections of very low doses [Harrison, L., et al., J. Eq. Vet Sci. 11:163-166(1991)] or a very high dose given as a slow releasing implant [Jochle, W. et al., J. Eq. Vet. Sci. 44:632(1994)].

Similar concerns and needs occur in other areas involving the raising and breeding of mammals, in particular livestock mammals, including, for example, the swine and cattle husbandry industries, where control of the estrus of sows and gilts and of cows and heifers, respectively, also would benefit their reproductive management.

In particular, in commercial swine production, maximizing reproductive efficiency offers producers substantial opportunities to reduce production costs and enhance profitability. Currently, a precise method of determining the time of ovulation in spontaneously cycling gilts is not available. In gilts and sows, GnRH affects the synchronization of ovulation. However, at present the variation in time of the onset of ovulation is large enough that two inseminations are required for maximal fertilization. Therefore, development of a controlled release GnRH formulation that could stimulate an LH surge capable of reducing the time span of ovulations so that a single timed insemination could be used would greatly benefit reproductive management of gilts, sows, and consequently, boars.

In cattle production, maximizing reproductive efficiency offers producers substantial opportunities to reduce production costs and enhance profitability. This is particularly true in the heifer, due to difficulties in synchronizing estrus compared with cows, a factor that reduces overall herd performance. Therefore, methods to synchronize estrus which increase the level of response and reduce variability would allow management to bring replacement heifers into the herd at lower cost and significantly impact the efficiency of beef and dairy production.

Progress toward reducing reliance on estrus detection for managing reproduction in dairy heifers and cows is being realized by combining timed artificial insemination (AI) with a protocol for synchronization of ovulation that can be iniated at a random stage of the estrous cycle. [Pursley, J. R., et al., Theriogenology 44:915-923 (1995); Pursley, J. R., et al., J. Dairy Sci. 80:295-300 (1997)]. This protocol, commonly called OVSYNCH, synchronizes follicular development, luteal regression and time of ovulation, thereby allowing for timed insemination 12 to 24 hours after the completion of the GnRH/PGF/GnRH treatment protocol. However, hormone cost per treated cow can be significant. [Frick, P. M., et al., Theriogenology 50:1275-1284 (1998)]. Since retail cost of GnRH constitutes the majority of the cost in using OVSYNCH, development of a cost effective controlled release GnRH formulation that could be used in the OVSYNCH protocol would greatly benefit reproductive management of heifers and cows.

In humans, GnRH or its analogs or agonists can be used to treat conditions related to the reproductive system. Examples include precocious puberty in children, endometriosis in women, prostate cancer in men, and other conditions requiring or responding to short term or long term GnRH therapy.

The selection of an appropriate drug delivery system should be based on the pharmacokinetic and pharmacodynamic properties of the drug. The importance of the pharmacodynamic properties of a drug is especially relevant in the case of hormones that target specific high affinity receptors to produce their effect. In the case of GnRH this relationship is dependent on multiple elements including species, reproductive status and complex concentration/presentation effects of the peptide and pituitary responsiveness to it.

### BRIEF DESCRIPTION OF THE INVENTION

Applicants have discovered that certain compositions are suitable for controlled release of GnRH, its analogs, and/or its agonists, particularly for the purpose of treating conditions relating to the reproductive system in a variety of species. In particular, the composition of the present invention can be used for advancing ovulation in domesticated animals, in particular in female mammals, and even more particularly, in mares, gilts, sows, ewes, cows, heifers, she-goats, and the like. The composition includes
(a) a non-polymeric, non-water soluble liquid carrier material having a viscosity of at least 5,000 cP at 37° C. that does not crystallize neat under ambient or physiological conditions;
(b) GnRH, analogs, or agonists, or a combination thereof.

In a particular embodiment, the composition includes a system based on sucrose acetate isobutyrate (SAIB) a fully-esterified sucrose molecule. SAIB is a low molecular weight material that has many of properties associated with polymeric materials. Because SAIB is a non-polymer, dilution with only small amounts of solvents are required to give an easily-injectable solution. Applicants have discovered a particular adaptation of the SAIB drug delivery system technology suitable for treating reproductive disorders susceptible to treatment by GnRH, in particular for inducing ovulation, in animals, and in particular in female mammals, e.g., mares, gilts and sows, ewes, cows, heifers, she-goats, and the like, with a composition that is injectable and sterilizable. In this particular embodiment, for inducing ovulation, the composition releases the GnRH or its analog or agonist over a relatively short time period, typically about 1 to about 12 hours, more particularly about 1 to about 6 hours.

The combination of non-polymeric, non-water soluble liquid carrier material, GnRH, or its analog or agonist, and solvent can be formulated as a solution or suspension of the GnRH in the solvent. Suspensions can be formed by, e.g., using a solvent in which GnRH, its analog or agonist is insoluble. Increased stability has been found to result from the use of a suspension.

In another particular embodiment, the composition can include additives that can substantially lengthen the delivery time up to several months, more particularly from about 1 to about 30 days, even more particularly from about 14 to about 30 days, thereby making the composition suitable for induction of cyclicity or ovulation in seasonally nonovulatory animals, such as mares, or for treatment of conditions susceptible to long term therapy with GnRH, or analogs or agonists thereof, such as precocious puberty in human children, endometriosis in women, and prostate cancer in men, and for inducing spawning in marine life, such as finned fish or shellfish.

In another embodiment, the invention relates to methods of treating reproductive disorders in animals by administering to an animal in need thereof an effective amount of the composition described above, i.e., a combination of non-polymeric, non-water soluble liquid carrier material and GnRH, or its analogs or agonists.

In yet another embodiment, the invention relates to methods of inducing ovulation in a female mammal by administering to the female mammal an effective amount of the composition described above, i.e., a combination of non-polymeric, non-water soluble liquid carrier material and GnRH, or its analogs or agonists. In particular embodiments of the invention, the female mammal may be a sow, gilt, cow, or heifer.

### Abbreviation and Definitions

GnRH Gonodotropin releasing hormone, also known as LH-RH or LHRH
HVLCM High viscosity liquid carrier material
LH Luteinizing Hormone
LH-RH Luteinizing Hormone-releasing Hormone
LVLCM Low viscosity liquid carrier material

### BRIEF DESCRIPTION OF THE FIGURES

FIG. 1 shows LH concentrations in mares following treatment with experimental formulations.

FIG. 2 shows deslorelin acetate concentrations in gilts following treatment with a formulation according to the invention.

FIG. 3 shows the LH concentrations in gilts following treatment with a formulation according to the invention.

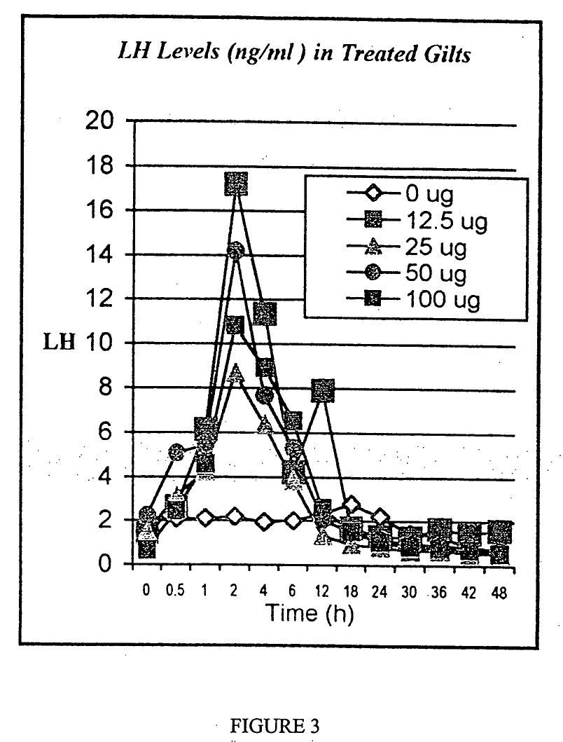

FIG. 4 shows the LH levels in heifers following treatment with a formulation according to the invention.

### DETAILED DESCRIPTION OF THE INVENTION

The present invention relates to a composition for the controlled release of GnRH or analogs or agonists thereof in animals, in particular in female mammals, and even more particularly, in domesticated female mammals, such as mares, gilts, and sows, cows, heifers, ewes, she-goats, bitches, cats, and the like to induce ovulation, comprising:
(a) a non-polymeric, non-water soluble liquid carrier material having a viscosity of at least 5,000 cP at 37° C. that does not crystallize neat under ambient or physiological conditions;
(b) GnRH or analogs, or combination thereof.

In one embodiment of the composition of the present invention, the non-water soluble liquid carrier material is sucrose acetate isobutyrate.

In another embodiment of the composition of the present invention, the non-water soluble liquid carrier material is present in an amount from about 99.5 percent to about 10 percent by weight, relative to the total weight of the composition.

In another embodiment of the composition of the present invention, the non-water soluble liquid carrier material is present in an amount from about 95 percent to about 25 percent by weight, relative to the total weight of the composition.

In another embodiment of the composition of the present invention, the composition further comprises a solvent in which the non-water soluble liquid carrier is soluble.

In another embodiment of the composition of the present invention, the solvent is selected from the group consisting of ethanol, dimethylsulfoxide, ethyl lactate, ethyl acetate, benzyl alcohol, triacetin, N-methylpyrrolidone, propylene carbonate, and glycofurol. The solvent may be one in which the GnRH, its analog or agonist, is insoluble, resulting in a suspension.

In another embodiment of the composition of the present invention, the solvent is propylene carbonate. In this embodiment, the GnRH, analog, or agonist may desirably be deslorelin, which is insoluble in propylene carbonate. The resulting composition forms a suspension.

In another embodiment of the composition of the present invention, the solvent is present in an amount from about 10 to about 50 percent by weight, relative to the weight of the composition.

In another embodiment of the composition of the present invention, the analog is deslorelin.

In another embodiment of the composition of the present invention, the analog is selected from deslorelin, avorelin, leuprolide, and natural LHRH.

The present invention also relates to a liquid composition for the controlled release of GnRH or analogs thereof in mares to induce ovulation, comprising sucrose acetate isobutyrate and ethanol in a weight ratio of between about 75:25 and about 60:40, and GnRH or analog thereof or combination thereof in a concentration of between about 0.1 to about 5.0 mg/ml of liquid composition, to provide a dose of between about 0.3 mg and about 10 mg of GnRH or analog thereof or combination thereof.

The present invention also relates to a liquid composition for the controlled release of GnRH or analogs thereof in mares to induce ovulation, comprising sucrose acetate isobutyrate and ethanol in a weight ratio of between about 75:25 and about 60:40, and GnRH or analog thereof or combination thereof in a concentration of between about 1.0 to about 2.5 mg/ml of liquid composition, to deliver a dose of between about 0.3 mg and about 11 mg of GnRH or analog thereof or combination thereof.

In one embodiment of the liquid compositions of the present invention, the analog of GnRH is deslorelin.

In another embodiment of the liquid compositions of the present invention, the composition is sterilized before administration to mares.

In another embodiment of the liquid compositions of the present invention, composition is filter sterilized before administration to mares.

The present invention also relates to a filter sterilized liquid composition for the controlled release of deslorelin in mares to induce ovulation, comprising sucrose acetate isobutyrate and ethanol in a weight to weight ratio of about 75:25, and deslorelin at a concentration of between about 0.1 and about 5.0 mg/ml of liquid composition, to deliver a dose between about 1 mg and about 2 mg of deslorelin, said composition administrable by injection.

The present invention also relates to a filter sterilized liquid composition for the controlled release of deslorelin in mares to induce ovulation, comprising sucrose acetate isobutyrate and ethanol in a weight to weight ratio of about 75:25, and deslorelin at a concentration of between about 1.0 and about 2.5 mg/ml of liquid composition, to deliver a dose between about 1 mg and about 2 mg of deslorelin, said composition administrable by injection.

The present invention also relates to a liquid composition, for the controlled release of deslorelin in gilts, sows, heifers, and cows to induce ovulation, comprising sucrose acetate isobutyrate and propylene carbonate in a weight ratio of about 70:30 and containing deslorelin acetate at a concentration about 25 μg/ml of liquid composition, to deliver a dose between about 12.5 and about 100 μg of deslorelin acetate, said composition adminstrable by injection. Of course, smaller doses of deslorelin acetate may be administered using, e.g., a concentration of about 12.5 μg/ml to achieve dosages of about 1 μg to about 12.5 μg. Desirably, dosages of between 1 μg and about 100 μg are administered, more particularly between about 6.25 μg and about 25 μg, even more particularly between about 6.25 μg and about 12.5 μg.

In embodiments of the invention wherein an additive is included in the composition to increase the delivery time for up to several months, more particularly up to 30 days, the composition can be used in methods for inducing spawning in marine animals, such as finned fish or shellfish.

I. High Viscosity Liquid Carrier Material (HVLCM)

A high viscosity liquid carrier material should be selected that is non-polymeric, non-water soluble, and has a viscosity of at least 5,000 cP, (and optionally at least 10,000, 15,000; 20,000; 25,000 or even 50,000 cP) at 37° C., and that does not crystallize neat under ambient or physiological conditions The term non-water soluble refers to a material that is soluble in water to a degree of less than one percent by weight under ambient conditions. Suitable carrier materials are disclosed in U.S. Pat. No. 5,747,058, the entire contents of which are hereby incorporated by reference.

In a preferred embodiment, the HVLCM significantly decreases in viscosity when mixed with a solvent to form a LVLCM that can be mixed with a substrate for controlled delivery. The LVLCM/substrate composition is typically easier to place in the body than a HVLCM/substrate composition, because it flows more easily into and out of syringes or other implantation means, and can easily be formulated as an emulsion. The LVLCM can have any desired viscosity. It has been found that a viscosity range for the LVLCM of less than approximately 1000 cP, and more particularly less than 200 cP, is typically useful for in vivo applications.

In a preferred embodiment, sucrose acetate isobutyrate (“SAIB”), a sucrose molecule esterified with two acetic acid and six isobutyric acid moieties, is used as the HVLCM. The structure of SAIB is set forth below.

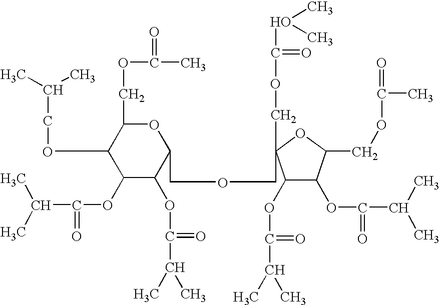

SAIB is orally non-toxic and is currently used to stabilize emulsions in the food industry. It is a very viscous liquid and has an unusual property that there is a dramatic change in viscosity with small additions of heat or with the addition of solvents. It is soluble in a large number of biocompatible solvents. When in solution or in an emulsion, SAIB can be applied via injection.

In other embodiments, the HVLCM can be stearate esters such as those of propylene glycol, glyceryl, diethylaminoethyl, and glycol, stearate amides and other long-chain fatty acid amides, such as N,N′-ethylene distearamide, stearamide MEA and DEA, ethylene bistearamide, cocoamine oxide, long-chain fatty alcohols, such as cetyl alcohol and stearyl alcohol, long-chain esters such as myristyl myristate, beheny erucate, and glyceryl phosphates. In a particular embodiment, the HVLCM is acetylated sucrose distearate (Crodesta A-10)

The HVLCM is present in the composition in any amount that achieves the desired affect. The HVLCM is typically present in controlled delivery compositions for GnRH or its analogs in an amount in the range from about 99.5 percent to about 10 percent by weight, more typically, between about 90 and about 25 percent, and most typically, between about 85 and about 65 percent, relative to the total weight of the composition.

II. Substance to be Delivered

A variety of analogs of gonodotrophlin releasing hormone are suitable for controlled release in the compositions of the present invention. Suitable analogs include, but are not limited to those listed in the following Table I.

TABLE I

| | Name |
|---|---|
| | (Commercial |
| Agonist structure | Source) |
| D-Trp⁶Pro⁹N Et LHRH | deslorelin |
| [Des-Gly¹⁰, D-2-Methyl-Trp⁶, Pro⁹-NHEt]-LHRH | Avorelin |
| | (Metrelin) |
| [D-Leu⁶, des-Gly-NH₂¹⁰]-LHRH (1-9) NHEt | Leuprolide |
| natural LHRH | (Abbott) |
| D-trp⁶-LHRH | triptorelin |
| | (Debioplarm) |
| | (Decapeptyn) |
| [D-Ser (But⁶des-GLy-NH₂¹⁰]-LHRK (1-9) NHEt | buserelin |
| | (Hoechst) |
| Des-Gly¹⁰-NH₂-LH-RH-ethylamide | fertirelin (Takeda) |
| [D-Trp⁶, MeLeu⁷, des-Gly-NH₂¹⁰]-LHRH(1-9)NHEt | Lutrelin (Wyeth) |
| [D-Ser(Bu^{t})⁶, Azgly¹⁰)-LHRH | Zoladex |
| | (Registered |
| | Trade Mark)ICI |
| [D-Ser(bu^{t})⁶,]-LHRH(1-9)NHEt | |
| [D-Lys(Boc)⁶, des-Gly-NH₂10]-LHRH(1-9)NHEt | |
| [D-Glu(OBu^{t})⁶, des-Gly-NH₂10]-LHRH(1-9)NHEt | |
| [D-Asp(OBu^{t})⁶, des-Gly-NH₂10]-LHRH(1-9)NHEt | |
| [D-Leu⁶Ser(Bu^{t}), des-Gly-NH₂10]-LHRH(1-9)NHEt | |
| [D-Ser(Bu^{t})⁶, Cys (Bu^{t})⁷des-Gly-NH₂10]-LHRH(1- | |
| [D-Ser(Bu^{t})⁶, Ser (Bu^{t})⁷des-Gly-NH₂10]-LHRH(1- | |
| [D-Phe⁶, Azgly¹⁰]-LHRH | |
| [D-Tyr(Me)⁶, Azgly¹⁰]-LHRH | |
| [D-Ser(Bu^{t})⁶, Azgly¹⁰]-LHRH | |
| [D-Tmo⁶] ]-LHRH | |
| [D-Nal(2)⁶]-LHRH | |
| [D-His (Benzyl)⁶, des-glyNH₂¹⁰]-LHRH(1-9)NHEt | historelin |

Preferred GnRH analogs include deslorelin, avorelin, leuprolide, and natural LHRH. Another series of preferred GnRH analogs includes triptorelin, nafarelin, goserelin, buserelin, and fertirelin. Most preferred is deslorelin.

The GnRH analogs are synthesized by any of a variety of conventional techniques. See generally Merrifield, B., Science 232:342 (1986), Norman, A. W. et al., Hormones Academic Press New York 1987. Deslorelin is synthesized by the method of Ajayaghosh, A. et al., J. Org. Chem. 55:2826(1990); Nestor, J. J. et al., Proc. Am. Pept. Symp. 7,109(1981); avorelin by the method of WO 91/18016; leuprolide by the methods of Ger. Pat. No. 2,446,005, U.S. Pat. No. 4,005,063; natural LHRH by the method of Ger. Pat. No. 2,213,737 and Coy et al. Methods Enzymol. 37, 416 (1975); triptorelin by the methods of Ger. Pat No. 2,625,843, U.S. Pat. No. 4,010,125; goserelin by the method of Ger. Pat. No. 2,720,245, U.S. Pat. No. 4,100,274; buserelin by the method of Ger. Pat. No. 2,438,352, U.S. Pat. No. 4,024,248; fertirelin by the method of Ger. Pat. No. 2,321,174; U.S. Pat. No. 3,853,837.

III. Solvent

When the composition is used as a LVLCM, it should contain a solvent in which the HVLCM is soluble. The substance to be delivered may also soluble in the solvent, however, increased stability of the composition may be obtained by forming a suspension of the substance to be delivered in the solvent. For example, increased stability has been obtained where the substance to be delivered is deslorelin by using propylene carbonate as the solvent, thereby forming a suspension of the deslorelin therein. The solvent should be non-toxic, water soluble or water miscible, and otherwise biocompatible. Solvents that are toxic should not be used for pharmaceutical or agricultural purposes. The solvents used to inject the composition into animals should not cause significant tissue irritation or necrosis at the site of implantation.

The solvent should be at least water soluble, so that it will diffuse quickly into bodily fluids or other aqueous environment, causing the composition to coagulate or solidify. Examples of suitable solvents include ethanol, ethyl lactate, propylene carbonate, glycofurol, N-methylpyrrolidone, 2 pyrrolidone, propylene glycol, acetone, methyl acetate, ethyl acetate, methyl ethyl ketone, benzyl alcohol, triacetin, dimethylformamide, dimethylsulfoxide, tetrahydrofuran, caprolactam, decylmethylsulfoxide, oleic acid, and l-dodecylazacycloheptan-2-one. A preferred solvent is propylene carbonate.

When SAIB is used as the HVLCM, the preferred solvents are ethanol, dimethylsulfoxide, ethyl lactate, ethyl acetate, benzyl alcohol, triacetin, N-methylpyrrolidone, propylene carbonate, and glycofurol. SAIB is not miscible with glycerol, corn oil, peanut oil, 1,2-propanediol, polyethylene glycol (PEG200), super refined sesame oil, and super refined peanut oil. Accordingly, the latter group of solvents are not preferred for use with SAIB.

The solvent is typically added to the compositions in an amount in the range from about 5 percent to about 55 percent by weight, relative to the total weight of the composition. Preferably, the solvent is present in the composition in an amount in the range from about 10 percent to about 50 percent by weight. Another preferred range is from about 10 percent to 30 percent by weight.

IV. Optional Additives

As described above, the delivery characteristics of the composition can be varied by adding one or more optional additives to the composition. These additives include those disclosed in U.S. Pat. No. 5,747,058, the entire contents of which are incorporated herein by reference. More particularly, suitable additives include biodegradeable polymers, non-biodegradeable polymers, natural oils, synthetic oils, carbohydrates or carbohydrate derivatives, inorganic salts, BSA (bovine serum albumin), surfactants, organic compounds, such as sugars, and organic salts, such as sodium citrate. In general, the less water soluble, i.e., the more lipophilic, the additive, the more it will decrease the rate of release of the substrate, compared to the same composition without the additive. In addition, it may be desirable to include additives that increase properties such as the strength or the porosity of the composition.

The addition of additives can also be used to lengthen the delivery time for the active ingredient (i.e., GnRH, its analogs or agonists), making the composition suitable for treatment of disorders or conditions responsive to longer term GnRH administration, as described above. Suitable additives in this regard include those disclosed in U.S. Pat. No. 5,747,058. In particular, suitable additives for this purpose include polymeric additives, such as cellulosic polymers and biodegradeable polymers. Suitable cellulosic polymers include cellulose acetates, cellulose ethers, and cellulose acetate butyrates. Suitable biodegradeable polymers include polylactic acid, polyglycolic acid, and copolymers thereof.

Additives, when present, are generally included in an amount ranging from about 0.02 wt % to about 20 wt %, more particularly from about 1 wt % to about 20 wt % based on the total weight of the composition.

V. Agricultural and Veterinary Uses of the LVLCM and HVLCM Compositions

The composition described herein can be administered to the host through a variety of methods which can vary depending on the result to be achieved. When the host is an animal, the composition can be administered, for example, topically, systematically (for example, mucosally (orally, rectally, vaginally, or nasally), or parenterally (intravenously, subcutaneously, intramuscularly, or intraperitoneally) in an appropriate carrier, if desired. Suitable hosts include female mammals, such as livestock or pet mammals, such as mares, gilts, sows, ewes, she-goats, cows, bitches, and the like.

Preferably, for veterinary purposes, the present compositions are administered as solutions or suspensions via injection. When administered via injection as a LVLCM, the small amount of solvent used in the composition leaches into the aqueous fluid of the host, forming a highly viscous depot for the controlled delivery of substances. See, for example, Ansel, H. C. et al., Pharmaceutical Dosage Forms and Drug Del. Systems, sixth ed., 1995.

### EXAMPLE 1

Preparation of SAIB Formulation 1.

A solution of deslorelin in DMSO (1.0 wt. %) was prepared. A concentrated solution of 95:5 weight ratio SAIB:DMSO was also prepared. A predetermined amount (2.1870 g) of deslorelin acetate (DA)/DMSO was added to 7.9230 g of the 95:5 SAIB:DMSO solution. The final formulation contained 2.4 mg/mL deslorelin and had an SAIB:DMSO ratio of 75:25.

Preparation of SAIB Formulation 2.

A solution of deslorelin in ethanol (2.1 wt. %) was prepared. A concentrated solution of 95:5 weight ratio SAIB:ethanol was also prepared. A predetermined amount (1.0376 g) of deslorelin acetate/ethanol was added to 8.9917 g of the 95:5 SAIB:ethanol solution. The final formulation contained 2.3 mg/mL deslorelin and had an SAIB:ethanol ratio of 85:15.

Preparation of SAIB Formulation 3.

A solution of deslorelin in ethanol (1.9 wt. %) was prepared. A concentrated solution of 95:5 weight ratio SAIB:ethanol was also prepared. A predetermined amount (1.0826 g) of deslorelin acetate/ethanol was added to 7.9085 g of the 95:5 SAIB:ethanol solution. The final formulation contained 2.2 mg/mL deslorelin and had an SAIB:ethanol ratio of 75:25.

Preparation of SAIB Formulations 4-8 with 75:25 SAIB:Ethanol for Dose Titration Study.

A dose titration study was performed using this 75:25 SAIB:ethanol formulation that evaluated deslorelin concentrations of 0.5, 1.0, 1.5, and 2.0 mg/mL. A concentrated solution of SAIB in ethanol (83.6 wt. %) was prepared and sterile filtered using a 0.2 μm hydrophobic filter. Pure ethanol and a solution of deslorelin in ethanol (21.0 mg/g) were sterile filtered using 0.22 μm sterile syringe filters.

Appropriate amounts of sterile SAIB/EtOH and EtOH/deslorelin solutions were combined with a predetermined amount of sterile ethanol to yield the final mixtures with the desired concentrations. The amounts of each component used and the compositions of the formulations prepared are shown in Table A. The lowest concentration of each formulation produced a solution, while the remaining formulations were suspensions that increased in cloudiness with increasing deslorelin concentration.

TABLE A
Compositions of 75:25 SAIB:Ethanol Formulations for In Vivo Study

| | | | | Amount of Component Added | | |
|---|---|---|---|---|---|---|
| Lot | Formula | SAIB/EtOH | DA | | | SAIB/ |
| Number | for # | Ratio | mg/ml | EtOH | EtOH/Des | EtOH |
| X96560 | 4 | 75:25 | 0.5 | 2.49 | 0.74 | 27.91 |
| X96561 | 5 | 75:25 | 1.0 | 1.79 | 1.49 | 28.39 |
| X96562 | 6 | 75:25 | 1.5 | 1.03 | 2.19 | 27.61 |
| X96563 | 7 | 75:25 | 2.0 | 0.31 | 2.91 | 27.64 |
| X96568 | 8 | 75:25 | 0 | 8.74 | — | 77.79 |

Preparation of SAIB Formulations 9-12 with 65:35 SAIB:Ethanol for Dose Titration Study.

A dose titration study was also performed using a 65:35 SAIB:ethanol formulation that evaluated deslorelin concentrations of 0.5, 1.0, 1.5, and 2.0 mg/mL. A concentrated solution of SAIB in ethanol (83.6 wt. %) was prepared and sterile filtered using a 0.2 μm hydrophobic filter. Pure ethanol and a solution of deslorelin in ethanol (21.0 mg/g) were sterile filtered using 0.22 μm sterile syringe filters.

Appropriate amounts of sterile SAIB/EtOH and EtOH/deslorelin solutions were combined with a predetermined amount of sterile ethanol to yield the final mixtures with the desired concentrations. The amounts of each component used and the compositions of the formulations prepared are shown in Table B. The lowest concentration of each formulation produced a solution, while the remaining formulations were suspensions that increased in cloudiness with increasing deslorelin concentration.

TABLE B
Compositions of 65:35 SAIB:Ethanol Formulations for In Vivo Study

| | | | | Amount of Component Added | | |
|---|---|---|---|---|---|---|
| Lot | Formula | SAIB/EtOH | DA | | | SAIB/ |
| Number | for # | Ratio | mg/ml | EtOH | EtOH/Des | EtOH |
| X96564 | 9 | 65:35 | 0.5 | 5.56 | 0.74 | 23.23 |
| X96565 | 10 | 65:35 | 1.0 | 4.98 | 1.43 | 22.29 |
| X96566 | 11 | 65:35 | 1.5 | 447 | 2.22 | 23.18 |
| X96567 | 12 | 65:35 | 2.0 | 3.64 | 2.17 | 22.60 |

### EXAMPLE 2

Mares used in this experiment were from the resident herd at the LSU Agricultural Center Horse Farm and were all of light horse type, mainly Quarter Horses, Thoroughbreds and Arabians. All mares were in good body condition and were maintained on native summer grass pasture (predominantly bermuda grass). The majority of mares in the herd were not bred the previous season, whereas six had foaled within 30 days and were lactating. The mares were placed on a daily regimen of estrous detection beginning June 1, and were all administered a general health and reproductive soundness exam during June. Only mares with good health, satisfactory vulvar and vaginal conformations, and apparently normal uterine and ovarian conformations were placed into a pool of potential candidates for treatment. Most of the mares were between 11 and 14 years of age (range: 8 to 22 years) and weighed 400 to 650 kg.

In this study, three experimental formulations were prepared by weighing and mixing SAIB (SABER, SBS Inc., Birmingham, Ala.), diluting solvent and deslorelin added to give a final concentration of 2.1 mg/ml. SAIB: diluting solvent compositions were: 75:25 w/w SAIB:DMSO in Formulation 1 (see example 1A); 85:15 w/w SAIB:Ethanol in Formulation 2 (see example 1B); and 75:25 w/w SAIB:Ethanol in Formulation 3 (see example 1C);. Resultant experimental formulations were hydrophobic low viscosity after i.m. injection as the solvent diffused, leaving behind a SAIB-DA matrix that released DA by diffusion through the highly viscous SAIB, accompanied by degradation of SAIB to sucrose and its corresponding aliphatic acids from which the sucrose ester was prepared. In addition, a negative control (1 mL of 0.9% NaCl USP, injected i.m.) was prepared. As mares entered estrus after July 1, their ovaries were evaluated daily by transrectal ultrasonography to assess follicular sizes and uterine appearance. Once a mare met the following two criteria, she was assigned to treatment based on a predetermined random allotment: 1) she had to be in estrus, and 2) she had to have a follicle of at least 30 mm in diameter, but not more than 40 mm diameter. Ultrasound evaluations were performed each morning, and mares were normally treated before noon. To avoid any possible biases in the data, the personnel administering treatments were different from those assessing follicular and estrous characteristics and injection sites. In addition, the three SABER formulations were color coded and their actual contents were unknown to all farm personnel.

Once a mare was treated, her ovaries were assessed via ultrasonography every 12 h until she ovulated. Sizes of the measurable follicles on each ovary were recorded, and ovulation was determined by various changes in size, softness, and the appearance of the dominant follicle as described in detail by Ginther (Ginther, O. J. Ultrasonic Imaging and Reproductive Events in the Mare Equiservices Cross Plains, Wis. 1986) In addition, blood samples were collected at 24 h before treatment-(−24 h); immediately before treatment (time 0); at 1, 3, 6, 12, 24, 36, and 48 h after treatment; and then every 24 h until 24 h after ovulation for measurement of progesterone and(or) luteinizing hormone (LH) concentrations. These blood samples were drawn via jugular venipuncture into heparinized, evacuated tubes, and the tubes were placed at 5C until plasma was harvested by centrifugation. Progesterone was measured by radioimmunoassay with commercially available reagents (Diagnostic Systems Laboratories, Inc., Webster, Tex.) and LH was measured by radioimmunoassay as described by Thompson et al. 1983. J. Anim. Sci. 56:678-686.

Each day after treatment for 7 days, the injection site of each mare was assessed for three characteristics: 1) swelling, which was scored as 0=none, 1=slight (1 cm diameter or less), 2=slight (1 to 2.5 cm diameter), and 3=significant (greater than 3 cm diameter); 2) sensitivity to touch, which was scored as yes or no; and 3) skin temperature elevation, which was also scored as yes or no. Deslorelin concentrations were determined in the blood samples collected at −24, 0, 1, 3, 6, 12, 24, 36, 48, and 72 h relative to treatment. Immediately after the sample was withdrawn from the jugular vein, a 1-mL aliquot was removed and added to 4 mL of acetone in a 12×75 mm disposable glass tube. This mixture was inverted several times and capped for storage at −15° C. At a later date, the extracts were centrifuged and the acetone decanted into a second tube. The acetone was then dried under a stream of air, and the residual aqueous solution was diluted back to 1.0 mL with assay buffer. Deslorelin was measured in the extracts by radioimmunoassay using an anti-GnRH antiserum (Rabb et al., 1990. J. Anim. Sci. 68:3322-3329) and radioiodinated deslorelin. The deslorelin was radioiodinated by the chloramine-T method and isolated by QAE-Sephadex chromatography as described for GnRH by Nett et al., Endocrinology 101:1135 (1977). Because endogenous GnRH is not present in sufficient quantities in jugular blood for detection, any immunoreactivity in the samples was assumed to be deslorelin and not GnRH.

During the course of the experiment, one mare exhibited an unusually long estrous period and did not ovulate until 216 h after treatment. Because her response was so different from all other mares, her ovulation time was compared to the remaining mares receiving deslorelin and was found to be 3.82 standard deviations away from their average (P<O1). Thus, the data from this mare were removed from all analyses, and an additional mare was treated with the slow release formulation (see example 1A) to take her place.

Data for single time points were analyzed by one-way analysis of variance using the General Linear Models procedure of SAS. 1988 SAS/STAT® User's Guide (Release 6.03), SAS Inst. Inc., Cary, N.C. For each variable, the comparison between the saline treated mares and all mares receiving deslorelin was included in the analysis, as well as individual comparisons between each group receiving deslorelin and the saline group; these comparisons were based on the LSD value calculated from the pooled error variance. For percentage of mares ovulating within 48 h, mares were scored either 1 (yes) or 0 (no) and a one-way analysis of variance was performed on those data rather than using the Chi-square method. Data from repeated sampling (e.g., LH concentrations) were analyzed by split-plot analysis of variance in which the effect of treatment was tested with the horse (treatment) term, and the treatment x time interaction was tested with the residual error variance. Net areas under the response curves were also calculated for deslorelin concentrations from 0 to 24 h after treatment, and for LH concentrations from 0 to 48 h after treatment; these areas were analyzed by one-way analysis of variance as described above.

Ovulation was confirmed by Ultrasound (US) and elevated progesterone (P₄) levels. End points studied included hours to ovulation, percentage (%) of mares ovulating within 48 h and LH, P₄ and DA levels measured by RIA's. In addition, injection site swelling scores (0 to 3; 0=none, 1=slight, 2=moderate, 3=significant), injection site sensitivity (Yes/No) and temperature elevation at the injection site (Yes/No) were also studied.

Results showed that plasma DA (deslorelin acetate) concentrations were significantly increased after treatment in all three SAIB formulations with peak levels of 1902 to 1699 pg/ml. Area under the response curve (AUC) for the first 24 hours after injection also confirmed significant increase in SAIB treated mares compared to saline treated controls (See Table II).

In Table II, a significant increase in the % of mares ovulating within 48 h (when compared with saline) was detected in mares given formulations 2 and 3.

TABLE II
Mare Efficacy & Hormonal Data

| | | |
|---|---|---|
| Hours to | | |
| Ovulation | Ovulation | DA AUC |

| | (US) | (P₄) | by 48 h | First 24 h |
|---|---|---|---|---|
| Saline Controls | 102 | 99 | 0% | 148.68 |
| Formulation 1 | 81 | 72 | 50% | 4983.20¹ |
| (example 1A) | | | | |
| Formulation 2 (1B) | 69 | 51² | 75²% | 6026.19¹ |
| Formulation 3 (1C) | 48³ | 48¹ | 100¹% | 6037.52¹ |

Safety - Injection Site (IS) Data

| | Swelling Score | | | | Sensitive | Temp. |
|---|---|---|---|---|---|---|
| | (0-3) on Days | | | | at IS | Elevation |
| | 1 | 3 | 5 | 7 | (%) | at IS (%) |
| Saline Controls | 0 | 0 | 0 | 0 | 0% | 0% |
| Formulation 1 | 0.25 | 0 | 0 | 0 | 0% | 0% |
| (example 1A) | | | | | | |
| Formulation 2 (1B) | .5 | .25 | .25 | 0 | 0% | 0% |
| Formulation 3 (1C) | .5 | 0 | 0 | 0 | 0% | 0% |
| | | | | | | (P < .01)¹; |
| | | | | | | (P < .05)²; |
| | | | | | | (P < .07)³ |

There was no effect of treatment (P=0.467) nor any interaction with time (P=0.817) for swelling scores at the injection site, nor was there any sensitivity or elevation of skin temperature at the injection site for any treatment.

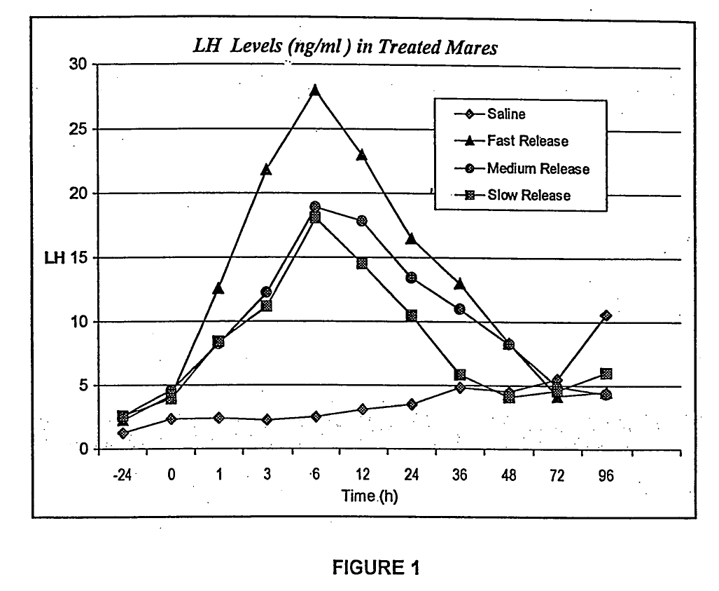

AUC for LH in the first 48 h, indicated that mares receiving Formulation 3 (P=0.01) and Formulation 2 (P=0.1) were increased compared to the saline treated mares. In addition, plasma LH concentration increased immediately (P<0.0003) in all treatments except saline, peaked at 6 h after injection and remained elevated through 36 to 48 h after treatment as shown in FIG. 1.

The results demonstrate that Formulations 2 and 3 effectively released deslorelin which stimulated ovulatory levels of LH and hastened ovulation in mares with 7 of 8 mares ovulating within 48 hours after treatment. Furthermore, the data inicate that all 3 SAIB formulations exhibited excellent biocompatibility as judged by minimal injection site reactions which were similar to controls.

### EXAMPLE 3

Ninety cyclic mares of various light horse breeds, 3 to 16 years old and weighing 400 to 650 kg were used. Mares were randomly assigned to one of 9 blinded color groups (n=10/group) to avoid interpretation bias. Treatments were 2 experimental formulation groups containing: 0.5, 1.0, 1.5 or 2.0 mg deslorelin acetate (DA) designed to deliver DA at differing rates for approximately 12 to 36 hours (h) after a 1 ml intramuscular (i.m.) injection using a 21 gauge needle; and a negative control consisting of SAIB containing no drug which was also administered as a 1 ml i.m. injection.

Experimental formulations were prepared by weighing and mixing SAIB (SABER, SBS Inc., Birmingham, Ala.), diluting solvent and DA added to give the appropriate final concentration of 0.5, 1.0, 1.5 or 2.0 mg/ml. SAIB: diluting solvent compositions were: 75:25 w/w SAIB:Ethanol in Formulations 4-8 (see example 1D) and 65:35 w/w SAIB:Ethanol in Formulations 9-12 (see example 1E).

Estrus mares' ovaries were examined daily by ultrasound (US) and were treated once a follicle between 30 mm and 40 mm was detected. Thereafter, mares' ovaries were examined every 24 h until ovulation which was confirmed by US.

The two major efficacy variables in the study were (a) interval in hours from treatment to ovulation, and (b) the percent of mares ovulating within 48 hours of treatment. The former was statistically analyzed using SAS® Cox's regression model (proportional hazards). The later was statistically analyzed using logistic regression investigating the effects of formulation and dose. The major safety variables in the study were (a) visible signs of swelling scores (b) sensitivity to touch, and (c) skin temperature elevation at the injection site. These variables were to be statistically analyzed by repeated measures analysis for categorical data using SAS PROC CATMOD, however, because no swelling, sensitivity or temperature elevations were detected the analysis was not performed.

Ovulation data are presented in Table III. Using the Cox model (linear) in doses for both formulation groups, the coefficient for the 75:25 SAIB/Ethanol formulation was highly significant (p<0.01 using a two sided test), but the coefficient for 65:35 SAIB/Ethanol formulations were not significant indicating the superiority of the 75:25 SAIB/Ethanol formulations (see example 1C).

TABLE III
Mare Ovulation Data
Control SAIB Formulation

| | Hours to | Mares Ovulating | |
|---|---|---|---|
| TREATMENTS | Ovulation | by 48 hours (%) | |
| Negative Control (0 mg) | 112.8 | 20% | (34%; 37%) |

75:25 SAIB/Ethanol Formulation

| TREATMENTS | Hours to | Mares Ovulating | |
|---|---|---|---|
| Example 1D | Ovulation | by 48 hours (%) | |
| Formulation 4 (0.5 mg) | 88.8 | 30% | (54%; 52%) |
| Formulation 5 (1.0 mg) | 50.4 | 90% | (74%; 70%) |
| Formulation 6 (1.5 mg) | 55.2 | 80% | (90%; 84%) |
| Formulation 7 (2.0 mg) | 50.4 | 90% | (98%; 92%) |

65:35 SAIB/Ethanol Formulation

| TREATMENTS | Hours to | Mares Ovulating | |
|---|---|---|---|
| Example 1E | Ovulation | by 48 hours (%) | |
| Formulation 9 (0.5 mg) | 60 | 70%* | (38%; 41%) |
| Formulation 10 (1.0 mg) | 74.4 | 50%* | (40%; 48%) |
| Formulation 11 (1.5 mg) | 110.4 | 50%* | (42%; 56%) |
| Formulation 12 (2.0 mg) | 79.2 | 60%* | (54; 44%) |

*Actual % (Cox's proportional hazards model-linear predicted %; logistics model predicted %) at (p < 0.01)

Using the logistic model (linear) in doses for both formulations, the effect of the 75:25 SAIB/ethanol formulations were also highly significant (p,0.01 using a two sided test), whereas the effect of the 65:35 SAIB/ethanol formulations were highly significant (p<0.1 using a two sided test). Moreover, the slope for the 75:25 formulations was significantly greater than that for the 65:35 formulations (B) (p=0.026) indicating the superiority of the 75:25 formulations.

Quadratic terms were not significant for either analysis indicating that the linear models used provided a sufficient representation for all nine groups. Predicted percentage of mares ovulating by 48 hours using both types of analysis are presented in table III in parenthesis next to the actual observed data.

The major safety variables in the study were visible signs of swelling, sensitivity to touch and skin temperature elevation at the injection site all of which were undetectable in the 90 mares studied. The absence of any observed swelling, sensitivity or elevation of skin temperature at the injection site of any of the treatments strong ly suggest excellent biocompatibility of the present SAIB formulation when produced using filter sterilization and administered using smaller 21 gauge needles (See Table IV).

TABLE IV
Mare Safety - Injection Site (IS) Data

| | Swelling Score | | | | | Temp. |
|---|---|---|---|---|---|---|
| TREATMENTS | (0-3) on Days | | | | Sensitivity | Elevation |
| Examples 1D & 1E | 1 | 3 | 5 | 7 | at IS (%) | at IS (%) |
| Negative Control (0 | 0 | 0 | 0 | 0 | 0% | 0% |
| Formulation 4 (0.5 mg) | 0 | 0 | 0 | 0 | 0% | 0% |
| Formulation 5 (1.0 mg) | 0 | 0 | 0 | 0 | 0% | 0% |
| Formulation 6 (1.5 mg) | 0 | 0 | 0 | 0 | 0% | 0% |
| Formulation 7 (2.0 mg) | 0 | 0 | 0 | 0 | 0% | 0% |
| Formulation 9 (0.5 mg) | 0 | 0 | 0 | 0 | 0% | 0% |
| Formulation 10 (1.0 | 0 | 0 | 0 | 0 | 0% | 0% |
| Formulation 11 (1.5 | 0 | 0 | 0 | 0 | 0% | 0% |
| Formulation 12 (2.0 | 0 | 0 | 0 | 0 | 0% | 0% |

This study clearly demonstrated the superiority of the 75:25 SAIB/ethanol formulation compared to the 65:35 SAIB/ethanol formulation for advancing ovulation. Furthermore, both Cox's proportional hazard and logistic modeling predicted a positive response rate for stimulating ovulation by 48 hours of greater than 70% for the 1 mg dose, 80% for the 1.5 mg dose and greater than 90% for the 2 mg dose of DA, indicating that such treatments effectively stimulate ovulatory levels of LH and hasten ovulation in the mare. Lastly, the safety data indicate that all nine SAIB formulations exhibited excellent biocompatibilty as judged by no detectable injection site reactions.

### EXAMPLE 4

Due to concerns about the stability of deslorelin as a solution it was decided to formulate deslorelin as a suspension using propylene carbonate as the diluting solvent because of deslorelin insolubility in propylene carbonate. Experimental formulations were prepared by weighing and mixing SAIB, solvent and desiorelin acetate (DA) to give a final concentration of 0.9 to 1.8 mg/ml. The SAIB: diluting solvent composition was:SAIB/propylene carbonate 70:30 w/w. The formulations were sterilized using gamma radiation.

In this Study, 30 cyclic mares of various light horse breeds, 3 to 16 years old and weighing 400 to 650 kg were used. Mares were randomly assigned to one of 2 blinded treatment groups (n=15/group) to avoid interpretation bias. Estrus mares were examined daily by transrectal ultrasound (US) and were treated once a follicle between 30 mm and 40 mm was detected. Mares were examined every 24 h thereafter, until ovulation. The efficacy endpoint studied was the percent of mares ovulating within 48 hours of treatment. Safety variables in the study were injection site swelling scores, sensitivity to touch, and skin temperature elevation at the injection site.

Examination of the data suggested that suspensions of deslorelin in SAIB/propylene carbonate worked well as for advancing ovulation in the mare. Observed results were that 100% (15/15) of the mares receiving the 1.8 mg group ovulated with in 48 hours and 80% (12/15) of the mares receiving the 0.9 mg group ovulated with in 48 hours. In addition, no mares were observed to experience swelling, sensitivity, or temperature elevations at the injection sites so safety and efficacy appeared to be similar to the previously studied solutions made with SAIB/ethanol. Based on the results of this study it was concluded that both doses prepared as a suspension using propylene carbonate as the diluting solvent provided good efficacy for advancing ovulation in the mare.

### EXAMPLE 5

Experimental formulations were prepared by weighing and mixing SAIB, propylene carbonate, and deslorelin acetate (DA) to give a final concentration of 25 μg/ml. The SAIB: propylene carbonate weight ratio was 70:30.

20 ovariectomized gilts approximately 200 days old and weighing about 100 kg were used. All gilts were ovariectomized for 15 to 30 days prior to entrance to the study. Prior to treatment, all gilts received estradiol benzoate at a dosage of 15 μg/kg. Forty eight hours after estradiol benzoate treatment, gilts were challenged with the deslorelin treatment. Treatments were designed to deliver DA for approximately 6 to 12 hours after i.m. injection of 0.5, 1, 2, or 4 mls delivering 12.5, 25, 50, or 100 μg, respectively. A control group (0 μg of DA) received 1 ml of the SAIB: propylene carbonate vehicle. Both treatment and control formulations were sterilized using gamma radiation.

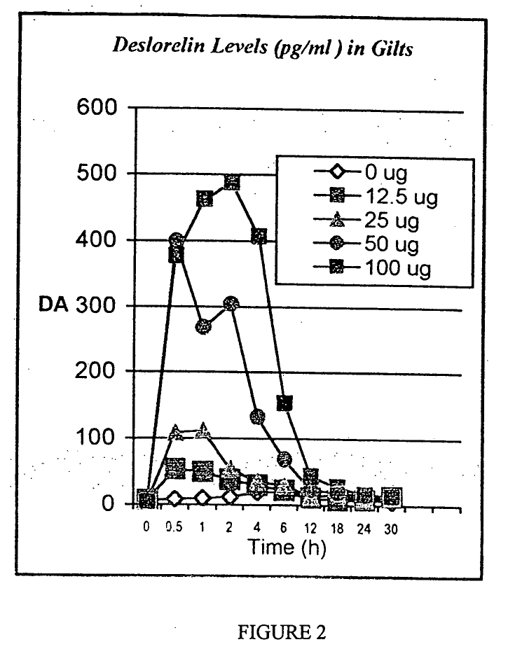

Cannulas were placed non-surgically into a jugular vein 24 hours prior to deslorelin treatment, and samples collected at 0, 0.5, 1, 2, 4, 6, 12, 18, 24, 30, 36, 42, and 48 hours after deslorelin treatment to determine the LH concentrations. Samples taken at 0, 0.5, 1, 2, 4, 6, 12, 18, 24, and 30 hours were used to determine DA concentrations. The levels of these compounds was determined by radioimmunoassay, and are provided graphically in FIGS. 2 (DA levels) and 3 (LH levels).

This data indicate that deslorelin levels were significantly elevated for 6 to 12 hours after DA treatment, with peak DA levels observed ranging from 53 to 487 μg/ml in the 12.5 and 100 μg groups, respectively. Serum LH concentrations were also significantly elevated in all of the DA treatment groups. Mean peak LH concentrations ranged from 12 ng/ml for gilts in the 25 μg group to 23 ng/ml for gilts in the 12.5 μg group. The lack of a dose response relationship is suggestive that the lowest dose of 12.5 μg was as capable of releasing stored pituitary LH as the higher doses studied. The lack of any decreased responsiveness for LH release at the higher doses studied also suggest that down-regulation of pituitary LH response does not occur with a single treatment of the formulation studied in doses of up to 100 μg, and that doses less than 12.5 μg may be effective. The magnitude of the LH peak is similar to previously reported peak ovulatory levels of 6 to 10 ng/ml in gilts.

### EXAMPLE 6

Experimental formulations were prepared by weighing and mixing SAIB, solvent and deslorelin acetate (DA) to give a final concentration of 25 μg/ml. The SAIB: diluting solvent composition was:SAIB/propylene carbonate 70:30 w/w). Experimental DA Treatments were sterilized using gamma radiation.

30 healthy cyclic dairy heifers weighing about 750-800 lbs (340-364 kg) were used. This study was designed as a parallel group design. All heifers were synchronized using pretreatment with 100 μg GnRH (Cystorellin, Merial, Ltd., Islin, NJ) on day −7 followed by 25 mg PGF₂α (Lutalyse Pharmacia-Upjohn Co. Kalamazoo, Mich.) treatment one week later. Forty eight hours after PGF₂α treatment heifers received saline (negative controls), Cystorellin (100 mg; positive controls) or Deslorelin-SABER (DA-slow delivery preparation; at 12.5, 25, 50 or 100 μg intra-muscular). Blood samples were collected by veni-puncture through shaved and aseptic-swabbed sites at −48, 0, 0.5, 1, 2, 4, 6, 12, 18, 24, 36, and 48 hours after treatment for LH analysis. Hefiers were randomly assigned to one of 6 blinded treatment groups (n=5/group) to avoid interpretation bias.

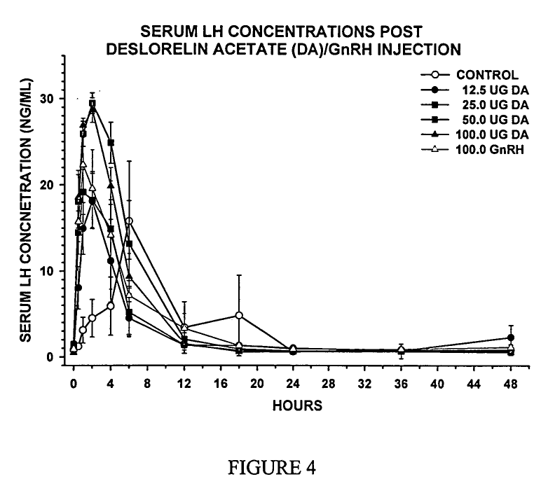

Statistical examination of the data indicated a significant treatment by time by dose interaction for LH concentrations. Results are illustrated in FIG. 4. Mean Peak LH concentrations ranged from 15.8 ng/ml in the saline treated control heifers at six hours post-treatment to 29.48 ng/ml in the 50 μg DA group at 2 hours post-treatment. LH concentrations in the heifers receiving the GnRH (100 μg) positive control treatment peaked at 1 hour post-treatment and averaged 22.3 ng/ml.

Analysis of area under the curve (AUC) suggested that maximal LH release occurred in the 50 μg group and 100 μg group. However, the data suggest that all groups, including the lowest dose of 12.5 μg, was as capable of releasing stored pituitary LH. The lack of any decreased responsiveness for LH release at the higher doses studied also suggest that down-regulation of pituitary LH response does not occur with a single treatment of the formulation studied at doses of up to 100 μg. These results suggest that the DA delivered as a SAIB/propylene carbonate suspension was effective at stimulating ovulatory levels of LH in the hefiers studied.

While the foregoing specification teaches the principles of the present invention, with examples provided for the purpose of illustration, it will be understood that the practice of the invention encompasses all of the usual variations, adaptations, or modifications, as come within the scope of the following claims and its equivalents.

What is claimed is:

1. A composition for the controlled release of GnRH, analogs or agonists thereof to treat disorders of the reproductive system or induce ovulation in animals, comprising:
(a) a non-polymeric, non-water soluble liquid carrier material having a viscosity of at least 5,000 cP at 37° C. that does not crystallize neat under ambient or physiological conditions;
(b) GnRH, analogs, agonists, or a combination thereof.

2. The composition of claim 1, wherein the non-water soluble liquid carrier material is sucrose acetate isobutyrate.

3. The composition of claim 2, wherein the non-water soluble liquid carrier material is present in an amount from about 99.5 percent to about 10 percent by weight, relative to the total weight of the composition.

4. The composition of claim 3, wherein the non-water soluble liquid carrier material is present in an amount from about 95 percent to about 25 percent by weight, relative to the total weight of the composition.

5. The composition of claim 2, wherein the composition further comprises a solvent in which the non-water soluble liquid carrier is soluble.

6. The composition of claim 5, wherein the solvent is selected from the group consisting of ethanol, dimethylsulfoxide, ethyl lactate, ethyl acetate, benzyl alcohol, triacetin, N-methylpyrrolidone, propylene carbonate, and glycofurol.

7. The composition of claim 5, wherein the solvent is propylene carbonate.

8. The composition of claim 5, wherein the solvent is present in an amount from about 10 to about 50 percent by weight, relative to the weight of the composition.

9. The composition of claim 1, wherein the GnRH analog is deslorelin.

10. The composition of claim 1, wherein the GnRH analog is selected from the group consisting of deslorelin, avorelin, leuprolide, and natural LHRH.

11. The composition of claim 1, wherein the composition is to induce ovulation in female mammals.

12. The composition of claim 11, wherein the mammal is a sow or gilt.

13. The composition of claim 5, comprising sucrose acetate isobutyrate and propylene carbonate in a weight ratio of about 70:30 and containing deslorelin acetate sufficient to deliver a dose of between about 1 μg and about 100 μg of deslorelin acetate.

14. The composition of claim 13, wherein the deslorelin acetate is present in the composition at a concentration of between about 12.5 μg/ml and about 25 μg/ml.

15. The composition of claim 14, wherein the deslorelin acetate is present in the composition at a concentration of about 25 μg/ml.

16. The composition of claim 13, wherein the dose of deslorelin acetate delivered is between about 6.25 μg and about 25 μg.

17. The composition of claim 16, wherein the dose of deslorelin acetate delivered is between about 6.25 μg and about 12.5 μg.

18. A method of treating reproductive disorders in animals, comprising administering to an animal in need thereof an effective amount of the composition of claim 1.

19. A method of inducing ovulation in a female mammal or spawning in a finned fish or shellfish, comprising administering to said female mammal, finned fish or shellfish an effective amount of the composition of claim 1.

20. The method of claim 19, wherein said female mammal is a sow or gilt.

21. The method of claim 20, wherein said female mammal is a cow or heifer.

22. The method of claim 21, wherein said female mammal is a heifer.

23. The method of claim 19, which comprises inducing cyclicity or ovulation in a seasonally nonovulatory mammal.

24. The method of claim 23, wherein said composition releases GnRH, analogs, or agonists thereof over a period of about 1 to about 30 days.

25. The method of claim 24, wherein said period is about 14 to about 30 days.

26. The method of claim 19, wherein said composition releases GnRH, analogs, or agonists thereof over a period of about 1 to about 6 hours.
